(19) United States
(12) Patent Application Publication
(10) Pub. No.: US 2023/0154589 A1
(43) Pub. Date: May 18, 2023

(54) Acupuncture and Moxibustion Treatment Support Apparatus, Acupuncture and Moxibustion Treatment Support Method, Acupuncture and Moxibustion Treatment Support Program, and Acupuncture and Moxibustion Treatment Support System

(71) Applicant: Seirin Corporation, Shizuoka (JP)

(72) Inventors: Ryoichi Nakano, Shimizu (JP); Masahiro Kikuchi, Shimizu (JP)

(73) Assignee: Seirin Corporation, Shizuoka (JP)

(21) Appl. No.: 17/912,673

(22) PCT Filed: Aug. 18, 2021
(86) PCT No.: PCT/JP2021/030147

§ 371 Date: Sep. 19, 2022

(30) Foreign Application Priority Data
Aug. 28, 2020 (JP) 2020-144578

(51) Int. Cl. G16H 20/30; G16H 10/60; G16H 15/00; G16H 20/90; G16H 40/20; G16H 70/20

(52) U.S. Cl. CPC G16H 20/30; G16H 10/60; G16H 15/00; G16H 20/90; G16H 40/20; G16H 70/20

(57) ABSTRACT

A treatment template, which is selected based on a symptom of a patient among treatment templates in each of which a symptom to be treated by acupuncture and moxibustion treatment is associated with treatment information indicating a treatment site for the symptom and a procedure to be performed at the treatment site, is read and obtained from a storage unit. Then, an electronic medical record of the patient to which the treatment information included in the obtained treatment template is applied is generated and stored in the storage unit.

### TECHNICAL FIELD

The present invention relates to an acupuncture and moxibustion treatment support apparatus, an acupuncture and moxibustion treatment support method, an acupuncture and moxibustion treatment support program, and an acupuncture and moxibustion treatment support system for supporting acupuncture and moxibustion treatment.

### BACKGROUND ART

In acupuncture and moxibustion treatment, an acupuncturist, who is a practitioner of acupuncture and moxibustion, identifies a symptom based on a primary complaint of a patient, determines a treatment method corresponding to the cause of the symptom, and provides appropriate treatment to alleviate the symptom of the patient. A technology for supporting acupuncture and moxibustion treatment has been proposed for making it possible to accurately identify the symptom of a patient. It has been proposed to have an information processing apparatus cause a display unit to display a human figure schematically illustrating a human body with site marks representing sites of the human body in the human figure, and when a site corresponding to a disorder of the patient is selected from the sites, cause the display unit to display a list of disorder patterns related to the selected site.

### REFERENCE DOCUMENT LIST

### Patent Document

Patent Document 1: JP 2019-079515 A

### SUMMARY OF THE INVENTION

### Problem to be Solved by the Invention

In acupuncture and moxibustion treatment, after identifying the symptom of a patient, an acupuncturist determines a treatment method suitable for the symptom. When determining a treatment method, the acupuncturist selects a procedure to be used in the treatment from a variety of procedures, and also selects a treatment site from many possible sites such as acupuncture points on the human body. Because there are diverse and complex combinations of procedures and treatment sites, highly specialized knowledge and a great deal of experience are necessary to determine a treatment method suitable for each patient.

For this reason, an object according to an aspect of the present invention is to make it possible to efficiently and accurately determine a treatment method and thereby improve treatment accuracy in acupuncture and moxibustion treatment.

### Means for Solving the Problem

According to one aspect of the present invention, a treatment template, which is selected based on a symptom of a patient among treatment templates in each of which a symptom to be treated by acupuncture and moxibustion treatment is associated with treatment information indicating a treatment site for the symptom and a procedure to be performed at the treatment site, is read and obtained from a storage unit. Then, an electronic medical record of the patient, to which the treatment information included in the obtained treatment template is applied, is generated and stored in the storage unit.

### Effects of the Invention

An aspect of the present invention makes it possible to efficiently and accurately determine a treatment method and thereby improve treatment accuracy in acupuncture and moxibustion treatment.

### BRIEF DESCRIPTION OF THE DRAWINGS

FIG. 1 is a block diagram illustrating an example of the overall configuration of an acupuncture and moxibustion treatment support system (first embodiment);

FIG. 2 is a drawing illustrating an example of a process performed by an acupuncture and moxibustion information setting unit and a treatment template setting unit;

FIG. 3 is a drawing illustrating an example of a settings screen;

FIG. 4 is a drawing illustrating an example of an acupuncture and moxibustion information list screen;

FIG. 5 is a drawing illustrating an example of an acupuncture and moxibustion information setting screen;

FIG. 6 is a drawing illustrating an example of a treatment template list screen;

FIG. 7 is a drawing illustrating an example of a treatment template setting screen (skeleton and skin);

FIG. 8 is a drawing illustrating an example of a treatment template setting screen (muscle);

FIG. 9 is a drawing illustrating an example of an acupuncture and moxibustion information selection screen;

FIG. 10 is a drawing illustrating an example of a process performed by a patient information setting unit, an electronic medical record setting unit, and a preliminary examination sheet setting unit;

FIG. 11 is a drawing illustrating an example of a patient list screen;

FIG. 12 is a drawing illustrating an example of a patient details screen;

FIG. 13 is a drawing illustrating an example of an electronic medical record setting screen;

FIG. 14 is a drawing illustrating an example of a treatment information setting screen;

FIG. 15 is a drawing illustrating an example of a treatment template selection screen;

FIG. 16 is a drawing illustrating an example of a preliminary examination sheet setting screen;

FIG. 17 is a block diagram illustrating an example of the overall configuration of an acupuncture and moxibustion treatment support system (second embodiment);

FIG. 18 is a drawing illustrating an example of a process performed by a treatment information analysis unit;

FIG. 19A is a drawing illustrating an example of a treatment information analysis screen;

FIG. 19B is a drawing illustrating an example of a treatment information analysis screen; and

FIG. 20 is a drawing illustrating an example of a hardware configuration of an information processing apparatus.

### MODES FOR CARRYING OUT THE INVENTION

### First Embodiment

### 1. Overall Configuration

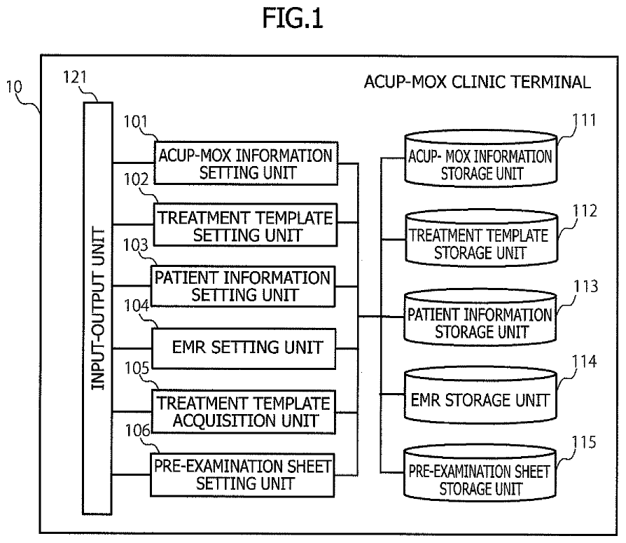

FIG. 1 illustrates an example of the overall configuration of an acupuncture and moxibustion treatment support system according to a first embodiment. The acupuncture and moxibustion treatment support system includes an acupuncture and moxibustion clinic terminal 10.

The acupuncture and moxibustion clinic terminal 10 is an information processing apparatus used at an acupuncture and moxibustion clinic that provides acupuncture and moxibustion treatment. The acupuncture and moxibustion clinic terminal 10 may be implemented by, for example, a desktop computer, a mobile computer, a tablet computer, a mobile device (e.g., a smartphone or PDA), or any other type of device. In the present embodiment, it is assumed that the acupuncture and moxibustion clinic terminal 10 is a tablet computer. An acupuncturist (a practitioner of acupuncture and moxibustion) is assumed to be the primary user of the acupuncture and moxibustion clinic terminal 10, and the present embodiment is described based on this assumption. However, the user of the acupuncture and moxibustion clinic terminal 10 is not limited to an acupuncturist.

The acupuncture and moxibustion clinic terminal 10 includes an acupuncture and moxibustion information setting unit 101, a treatment template setting unit 102, a patient information setting unit 103, an electronic medical record setting unit 104, a treatment template acquisition unit 105, and a preliminary examination sheet setting unit 106, the functions of which are implemented by executing an acupuncture and moxibustion treatment support program on the acupuncture and moxibustion clinic terminal 10. Also, the acupuncture and moxibustion clinic terminal 10 includes a storage device (storage unit) that includes an acupuncture and moxibustion information storage unit 111, a treatment template storage unit 112, a patient information storage unit 113, an electronic medical record storage unit 114, and a preliminary examination sheet storage unit 115. Furthermore, the acupuncture and moxibustion clinic terminal 10 includes an input-output unit 121.

Below, the major functions of the acupuncture and moxibustion information setting unit 101, the treatment template setting unit 102, the patient information setting unit 103, the electronic medical record setting unit 104, the treatment template acquisition unit 105, and the preliminary examination sheet setting unit 106 are described.

The acupuncture and moxibustion information setting unit 101 stores, in the acupuncture and moxibustion information storage unit 111, acupuncture and moxibustion information that is entered via a user interface displayed on a screen (touch panel) of a tablet functioning as both an input unit and an output unit and that includes procedures in acupuncture and moxibustion treatment and details of acupuncture and moxibustion products used in the procedures. The acupuncture and moxibustion information stored in advance in the acupuncture and moxibustion information storage unit 111 is used when treatment templates and electronic medical records are entered, as described later.

The treatment template setting unit 102 receives, via a user interface, input of a symptom that is the subject of acupuncture and moxibustion treatment and treatment information that includes a treatment site corresponding to the symptom, a procedure performed at the treatment site, and acupuncture and moxibustion information corresponding to the procedure. Then, based on the input information, the treatment template setting unit 102 generates a treatment template in which a treatment template name and the treatment information are associated with each other, and stores the treatment template in the treatment template storage unit 112. Here, a symptom in the present embodiment indicates information that also includes a site with, for example, pain. In the present embodiment, information indicating a symptom is included in the treatment template name.

The patient information setting unit 103 sets, in the patient information storage unit 113, basic information about a patient that is entered via a user interface and includes, for example, the name, the sex, the age, the occupation, and the contact information of the patient. Normally, the patient information is set when a new patient visits an acupuncture and moxibustion clinic.

The electronic medical record setting unit 104 generates an electronic medical record of a patient when acupuncture and moxibustion treatment is provided to the patient, and stores the electronic medical record in the electronic medical record storage unit 114. An electronic medical record includes, for example, patient information of a patient and a symptom and treatment information of the patient that are entered via a user interface. When generating an electronic medical record, the electronic medical record setting unit 104 may apply, to the electronic medical record, treatment information included in a treatment template obtained by the treatment template acquisition unit 105, described later. Also, the electronic medical record setting unit 104 may apply, to an electronic medical record, information on a symptom of a patient included in a preliminary examination sheet that is stored in the preliminary examination sheet storage unit 115 by the preliminary examination sheet setting unit 106, described later.

The treatment template acquisition unit 105 displays a list of treatment templates stored in the treatment template storage unit 112 on a screen, and reads and obtains, from the treatment template storage unit 112, a treatment template that is selected by the acupuncturist from the treatment templates based on a symptom of a patient.

The preliminary examination sheet setting unit 106 generates, based on information indicating a symptom of a patient before treatment and entered via a user interface, a preliminary examination sheet that includes the information and is unique to the patient, and stores the preliminary examination sheet in the preliminary examination sheet storage unit 115.

Next, data stored in the acupuncture and moxibustion information storage unit 111, the treatment template storage unit 112, the patient information storage unit 113, the electronic medical record storage unit 114, and the preliminary examination sheet storage unit 115 is described.

The acupuncture and moxibustion information storage unit 111 stores procedures used in treatment, and acupuncture and moxibustion information. Specifically, the acupuncture and moxibustion information storage unit 111 stores procedure types (for example, “retaining needle”, “electrification”, “moxa needle”, “press tack needle”, “single needle”, “moxibustion”, etc.) and as an example of acupuncture and moxibustion information, types of acupuncture and moxibustion products, acupuncture needle numbers (indicating thicknesses), lengths of acupuncture needles, and needle insertion angles and depths used in procedures.

The treatment template storage unit 112 stores information on treatment templates. For example, a treatment template includes a treatment template name, a treatment site, and a procedure and acupuncture and moxibustion information for the treatment site. Treatment templates stored in the treatment template storage unit 112 may include both treatment templates generated and stored by the treatment template setting unit 102 and treatment templates generated externally in advance and stored by, for example, an operation performed by a system administrator (e.g., treatment templates generated in advance by the system administrator when the acupuncture and moxibustion treatment support system is provided, and treatment templates newly provided during the operation of the system).

In the patient information storage unit 113, patient information, which is basic information about patients, is stored in advance. The patient information includes, for example, name, sex, age, occupation, and contact information of patients.

The electronic medical record storage unit 114 stores information of electronic medical records of individual patients. For example, an electronic medical record includes patient information such as the name of a patient, treatment date, acupuncturist in charge, symptom of the patient, treatment site, and procedure and acupuncture and moxibustion information for the treatment site.

The preliminary examination sheet storage unit 115 stores information on preliminary examination sheets of individual patients. For example, a preliminary examination sheet includes patient information such as the name of a patient, symptom of the patient before treatment, medical history, and lifestyle.

The input-output unit 121 is implemented by, for example, various drivers that function as interfaces for an input device and an output device included in the acupuncture and moxibustion clinic terminal 10.

### 2. Processes Performed by the Program

Next, the processes performed by the acupuncture and moxibustion information setting unit 101, the treatment template setting unit 102, the patient information setting unit 103, the electronic medical record setting unit 104, the treatment template acquisition unit 105, and the preliminary examination sheet setting unit 106 are described in detail with reference to examples of screens displayed in the processes. Basic functions of the acupuncture and moxibustion treatment support program of the present embodiment include a function for making various settings that can be applied to all patients in common and a function for setting and using information such as electronic medical records related to individual patients.

### 2.1. Process Related to the Function of Making Various Settings

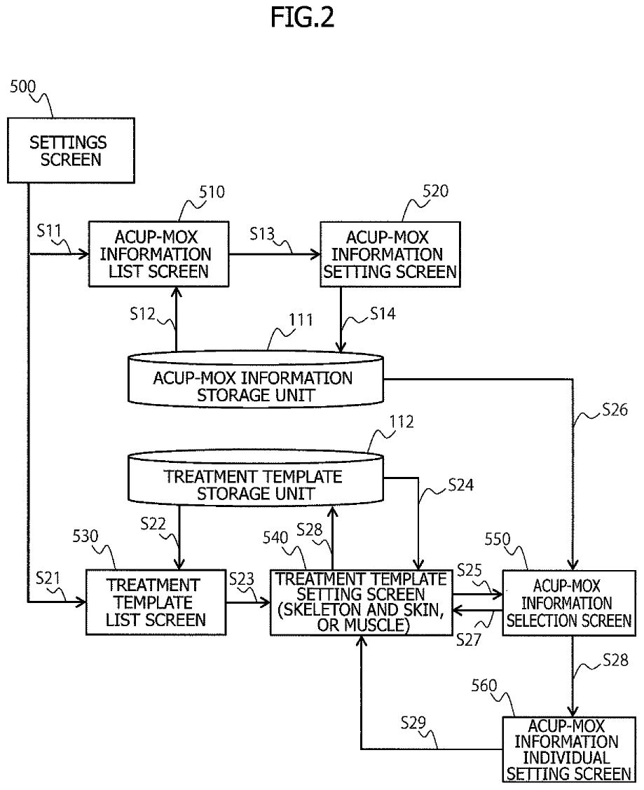

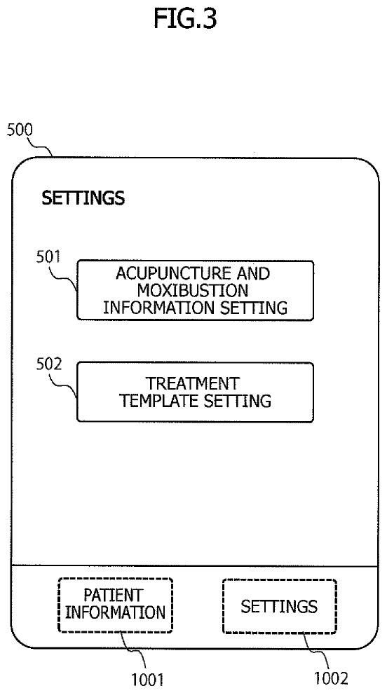

First, among the above-described basic functions, the function for making various settings applicable to all patients in common is described. FIG. 2 is a drawing illustrating an example of a process that is related to the function for making various settings and performed by the acupuncture and moxibustion information setting unit 101 and the treatment template setting unit 102, along with screen transitions and access to storage units. FIG. 3 illustrates a state in which an example of a settings screen 500, which is one of the basic screens indicating the basic functions described above, is displayed. In each basic screen, a patient information screen link 1001 and a settings screen link 1002 are displayed at the bottom as user interfaces for enabling transitions between basic screens implementing different basic functions. All of the types of user interfaces (for example, links, buttons, text boxes, pull-down menus, etc.) on screens explained in this specification are just examples, and the types of user interfaces are not limited to those used in this specification.

The settings screen 500 displays, in addition to the links to the basic screens, an acupuncture and moxibustion information setting button 501 for transitioning to an acupuncture and moxibustion information list screen 510 and a treatment template setting button 502 for transitioning to a treatment template list screen 530.

### 2.1.1. Acupuncture and Moxibustion Information Setting Process

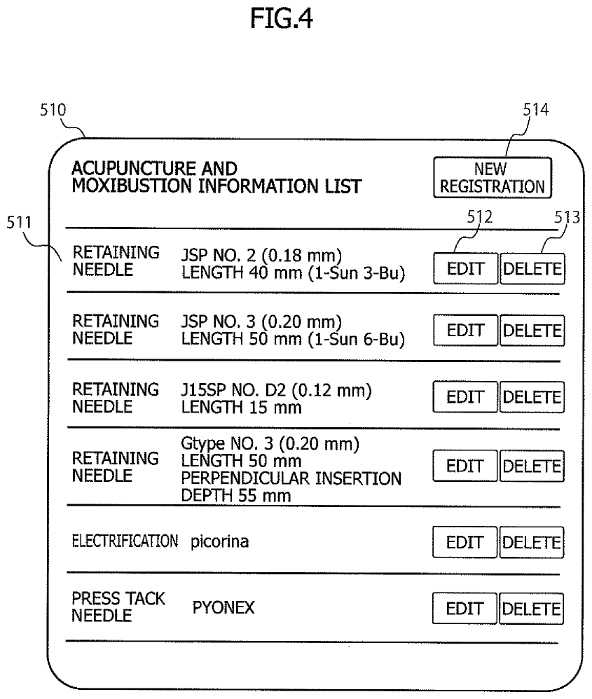

When the acupuncture and moxibustion information setting button 501 is pressed on the settings screen 500 by an operation by the acupuncturist, the acupuncture and moxibustion information setting unit 101 displays the acupuncture and moxibustion information list screen 510 illustrated in FIG. 4 (S11 in FIG. 2). Also, the acupuncture and moxibustion information setting unit 101 reads acupuncture and moxibustion information from the acupuncture and moxibustion information storage unit 111 and displays the read information as an acupuncture and moxibustion information list 511 on the acupuncture and moxibustion information list screen 510 (S12). Each row of acupuncture and moxibustion information in the acupuncture and moxibustion information list 511 displays an edit button 512 for updating the corresponding acupuncture and moxibustion information and a delete button 513 for deleting the corresponding acupuncture and moxibustion information. Furthermore, the acupuncture and moxibustion information list screen 510 displays a new registration button 514 for registering new acupuncture and moxibustion information.

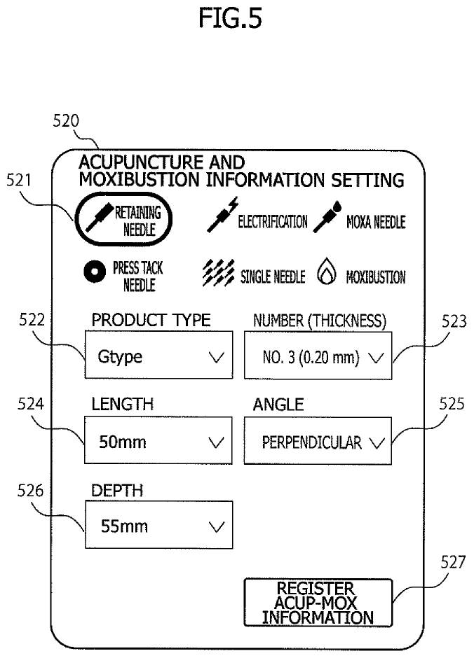

When the new registration button 514 of the acupuncture and moxibustion information list screen 510 is pressed by the user, the acupuncture and moxibustion information setting unit 101 displays an acupuncture and moxibustion information setting screen 520 as illustrated in FIG. 5 (S13). For example, the acupuncture and moxibustion information setting screen 520 displays a procedure selection field 521 for selecting a procedure and displays “retaining needle”, “electrification”, “moxa needle”, “press tack needle”, and “single needle” as selectable procedures. Furthermore, the acupuncture and moxibustion information setting screen 520 displays an acupuncture and moxibustion product type 522, a number (thickness) 523, a length 524, and an angle 525 and a depth 526 of needle insertion as input fields for entering acupuncture and moxibustion information corresponding to the selected procedure. The input field of the acupuncture and moxibustion product type 522 is configured such that types corresponding to a procedure selected in the procedure selection field 521 become selectable. For example, when “retaining needle” is selected as a procedure, various types of acupuncture needles become selectable; and when “electrification” is selected, various types of electrification devices become selectable. Similarly, information that can be entered in each of the number (thickness) 523, the length 524, the angle 525, and the depth 526 changes depending on the type of acupuncture and moxibustion product selected in the acupuncture and moxibustion product type 522. For example, when “retaining needle” is selected as a procedure, information can be entered in all of the input fields including the acupuncture and moxibustion product type 522, the number (thickness) 523, the length 524, the angle 525, and the depth 526. In contrast, when “press tack needle” is selected, information can be entered only in the acupuncture and moxibustion product type 522, the number (thickness) 523, and the length 524. Also, for example, the acupuncture and moxibustion information setting screen 520 may be configured such that when “electrification” is selected, types of input fields change to enable entry of, for example, an electrification time.

Here, entry of acupuncture and moxibustion information is not essential, and when a treatment site is specified, only a procedure to be performed at the treatment site may be selected. Also, the procedures displayed in the procedure selection field 521 of the present embodiment are just examples, and procedures are not limited to these examples. Similarly, acupuncture and moxibustion information is not limited to the information entered in the fields described above and may include any other types of information. Furthermore, the input fields of acupuncture and moxibustion information described above are just design examples, and all or some of the input fields may be configured, for example, to allow the acupuncturist to manually enter information.

The acupuncture and moxibustion information setting screen 520 further displays an acupuncture and moxibustion information registration button 527 for registering the procedure and the acupuncture and moxibustion information that are entered. Here, each of the screens including the acupuncture and moxibustion information setting screen 520 includes a cancellation function to cancel the corresponding process and a function to return to the previous screen. Descriptions and illustration of such functions are omitted in this specification and the drawings.

When the acupuncture and moxibustion information registration button 527 is pressed on the acupuncture and moxibustion information setting screen 520, the acupuncture and moxibustion information setting unit 101 stores, in the acupuncture and moxibustion information storage unit 111, the procedure selected in the procedure selection field 521 on the acupuncture and moxibustion information setting screen 520 and the acupuncture and moxibustion information entered in the input fields including the acupuncture and moxibustion product type 522, the number (thickness) 523, the length 524, the angle 525, and the depth 526 (S14).

Although not illustrated in FIG. 2, when the edit button 512 is pressed in any row of acupuncture and moxibustion information in the acupuncture and moxibustion information list 511 on the acupuncture and moxibustion information list screen 510, the acupuncture and moxibustion information setting screen 520 is displayed in a state in which the corresponding acupuncture and moxibustion information is entered in the input fields. Then, by changing the entered information on this screen and pressing the acupuncture and moxibustion information registration (change) button 527, the acupuncture and moxibustion information stored in the acupuncture and moxibustion information storage unit 111 can be updated as necessary. Also, by pressing the delete button 513 in any row of acupuncture and moxibustion information in the acupuncture and moxibustion information list 511, the corresponding acupuncture and moxibustion information can be deleted from the acupuncture and moxibustion information storage unit 111.

### 2.1.2. Treatment Template Setting Process

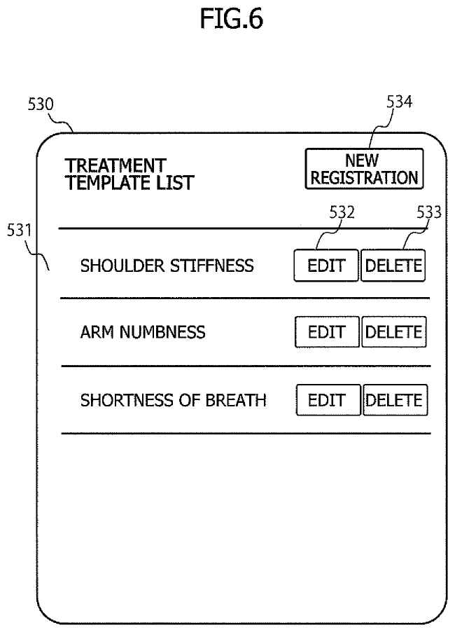

When the treatment template setting button 502 is pressed on the settings screen 500 by a user operation, the treatment template setting unit 102 displays a treatment template list screen 530 illustrated as an example in FIG. 6 (S21 in FIG. 2). In this step, the treatment template setting unit 102 reads treatment templates from the treatment template storage unit 112 and displays the read treatment templates as a treatment template list 531 on the treatment template list screen 530 (S22). Each row of acupuncture and moxibustion information in the treatment template list 531 displays an edit button 532 for updating the corresponding treatment template and a delete button 533 for deleting the corresponding treatment template. Furthermore, the treatment template list screen 530 displays a new registration button 534 for registering a new treatment template.

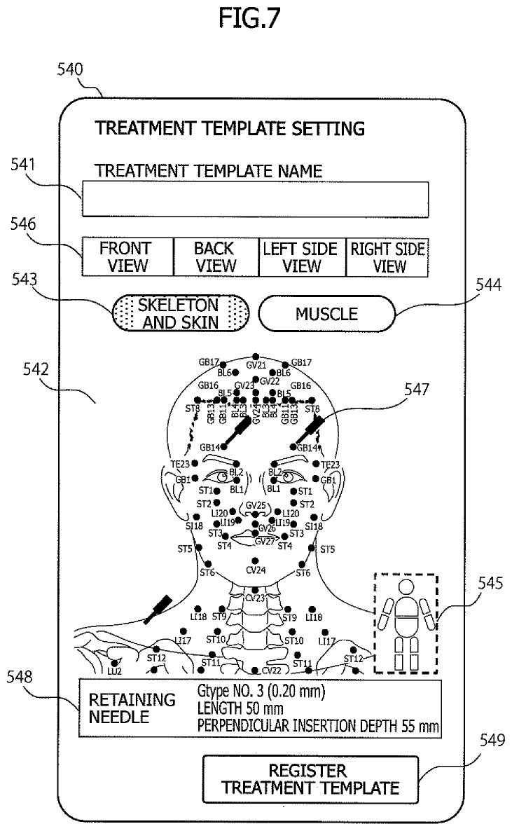

When the new registration button 534 on the treatment template list screen 530 is pressed by the user, the treatment template setting unit 102 displays a treatment template setting screen 540 illustrated as an example in FIG. 7 (S23).

The treatment template setting screen 540 displays a treatment template name 541 that is an input field for entering a name of a treatment template and a treatment input field 542 for entering, on a human figure, treatment information indicating a treatment site and a procedure performed at the treatment site. The acupuncturist enters, in the treatment template name 541, the name of a treatment template to be set. Here, it is assumed that information indicating a symptom, which is one of the symptoms that can be treated with acupuncture and moxibustion and is the subject of treatment information to be set by the treatment template, is included in the treatment template name. The information indicating a symptom may be any type of information (e.g., a serial number) and may not explicitly indicate the symptom, as long as the information can be used by the user to identify, in some way, the symptom for which the treatment template is generated.

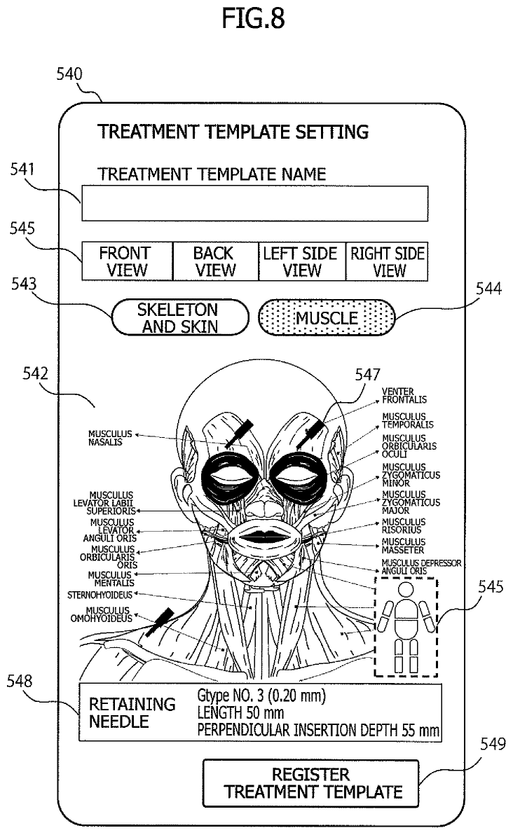

Next, the acupuncturist enters a treatment site and a procedure performed at the treatment site on the human figure in the treatment input field 542. Although multiple treatment sites generally exist, the present invention is not limited to this example. In the treatment input field 542, by pressing a skeleton and skin button 543 or a muscle button 544, it is possible to switch between a display of a human figure representing a skeleton and skin as illustrated in FIG. 7 and a display of a human figure representing muscles as illustrated in FIG. 8. Also, in the treatment input field 542, when a part of a human body is tapped at a human body mark 545, a portion of the human figure corresponding to the tapped part is enlarged. Furthermore, in the treatment input field 542, it is possible to change the orientation of the human figure being displayed by pressing one of display orientation switching buttons 546 that specify the orientations (front view, back view, left side view, or right side view) of the human figure.

When the user specifies a treatment site on the human figure by an operation such as tapping, for example, an icon 547 for “retaining needle” is displayed as a default value at the specified treatment site. When the human figure represents a skeleton and skin, and acupuncture points are displayed as illustrated in FIG. 7, the user can specify any of the acupuncture points as a treatment site. Here, even when the acupuncture points are displayed, the user can specify any position other than the acupuncture points. Also, when the human figure represents muscles as illustrated in FIG. 8, the user can specify any position as a treatment site, regardless of whether it is an acupuncture point, while referring to the positions of muscles. The treatment template setting unit 102 can use the name of an acupuncture point as information indicating a treatment site when the treatment site matches the acupuncture point and use, for example, coordinate information on the human figure as information indicating a treatment site when the treatment site does not match any acupuncture point. The coordinate information may be represented by numerical values indicating an absolute position in the entire human figure or by numerical values indicating, for example, a relative position relative to the position of an acupuncture point around the treatment site. With these two types of user interfaces, the acupuncturist can flexibly specify a treatment site according to a method suitable for the acupuncturist's treatment policy.

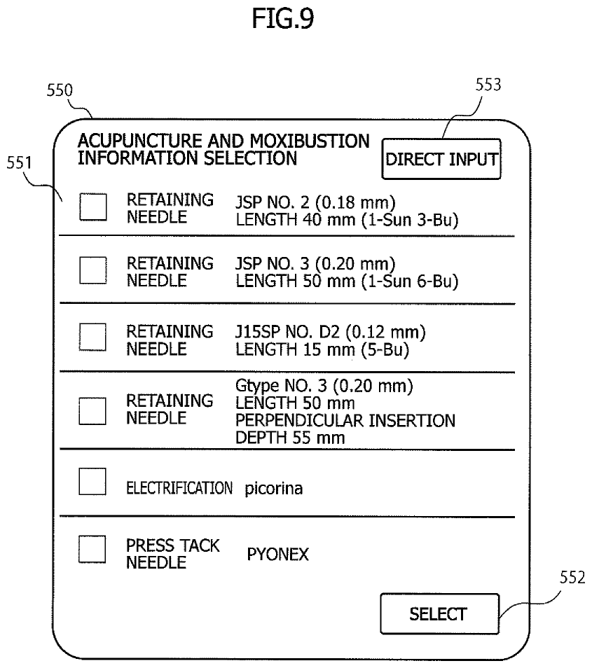

Here, when the icon 547 of “retaining needle” is displayed as a default value, one of multiple sets of acupuncture and moxibustion information stored in the acupuncture and moxibustion information storage unit 111 is displayed as default values in an acupuncture and moxibustion information field 548 at the bottom of the screen (S24). Default values of the icon 547 and the acupuncture and moxibustion information field 548 may be set at the same time as the acupuncture and moxibustion information is set by the acupuncture and moxibustion information setting unit 101. When it is desired to set a procedure and acupuncture and moxibustion information different from the default values as treatment information, the acupuncturist selects the acupuncture and moxibustion information field 548 by an operation such as tapping. In response, the treatment template setting unit 102 displays an acupuncture and moxibustion information selection screen 550 illustrated as an example in FIG. 9 (S25). Then, the treatment template setting unit 102 reads acupuncture and moxibustion information stored in the acupuncture and moxibustion information storage unit 111 and displays the read acupuncture and moxibustion information as acupuncture and moxibustion information selection candidates 551 on the acupuncture and moxibustion information selection screen 550 (S26). When the acupuncturist selects, in the acupuncture and moxibustion information selection candidates 551, a check box corresponding to acupuncture and moxibustion information to be included in treatment information and presses a selection button 552, the treatment template setting unit 102 displays the treatment template setting screen 540 again (S27), and displays the selected (specified) acupuncture and moxibustion information in the acupuncture and moxibustion information field 548. Also, when it is desired to set, in a treatment template, acupuncture and moxibustion information other than the acupuncture and moxibustion information stored in the acupuncture and moxibustion information storage unit 111, the acupuncturist presses a direct input button 553 on the acupuncture and moxibustion information selection screen 550. Then, the treatment template setting unit 102 displays an acupuncture and moxibustion information individual setting screen 560 (S28). Here, because the acupuncture and moxibustion information individual setting screen 560 has the same configuration as the acupuncture and moxibustion information setting screen 520, its illustration is omitted. When acupuncture and moxibustion information is entered on the acupuncture and moxibustion information individual setting screen 560 and an acupuncture and moxibustion information registration (setting) button is pressed, the treatment template setting screen 540 is displayed again (S29), and the entered acupuncture and moxibustion information is displayed in the acupuncture and moxibustion information field 548.

When the icon 547 is specified again at the treatment site for which treatment information has already been entered, the acupuncture and moxibustion information entered at steps S25 through S29 is displayed in the acupuncture and moxibustion information field 548. The displayed acupuncture and moxibustion information can be further changed.

When the user completes all entries on the treatment template setting screen 540 and presses a treatment template registration button 549, the treatment template setting unit 102 generates a treatment template including the treatment template name, the treatment sites, and the procedures and the acupuncture and moxibustion information for the respective treatment sites entered on the treatment template setting screen 540 (also including information entered on the acupuncture and moxibustion information selection screen 550 and the acupuncture and moxibustion information individual setting screen 560), and stores the generated treatment template in the treatment template storage unit 112 (S28).

Although not illustrated in FIG. 2, when the edit button 532 is pressed in each row representing a treatment template in the treatment template list 531 on the treatment template list screen 530, the treatment template setting screen 540 is displayed with the information items of the treatment template entered in the corresponding input fields. Then, by changing the entered information items on the treatment template setting screen 540 and pressing an acupuncture and moxibustion information registration (change) button 549, the treatment template stored in the treatment template storage unit 112 can be updated freely. Also, by pressing the delete button 533 in each row representing a treatment template in the treatment template list 531, the treatment template can be deleted from the treatment template storage unit 112.

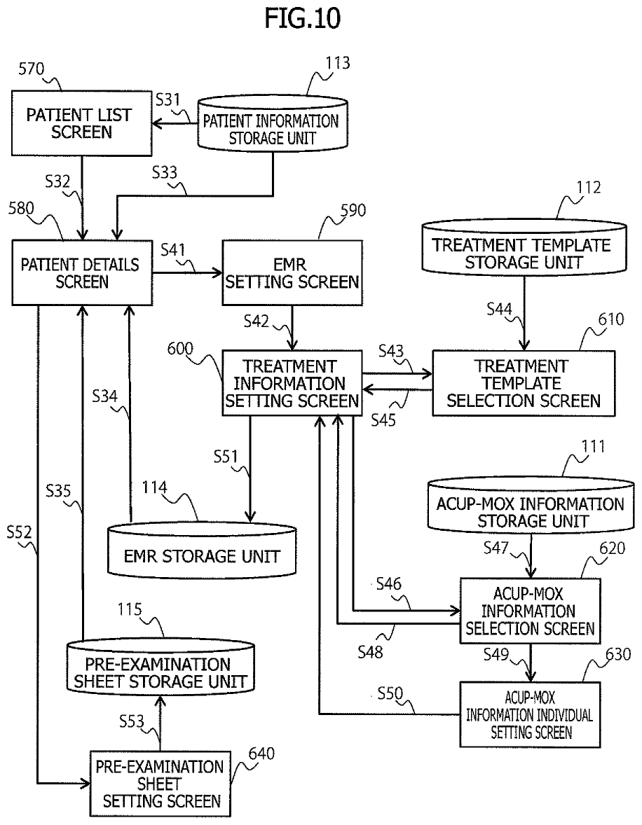

2.2. Function for Setting Information for Individual Patients Next, among the basic functions described above, a function for setting information on individual patients is described. FIG. 10 illustrates a process performed by the patient information setting unit 103, the electronic medical record setting unit 104, the treatment template acquisition unit 105, and the preliminary examination sheet setting unit 106 that are related to the function for setting information on individual patients, along with screen transitions and access to storage units.

### 2.2.1. Patient Information Setting Process

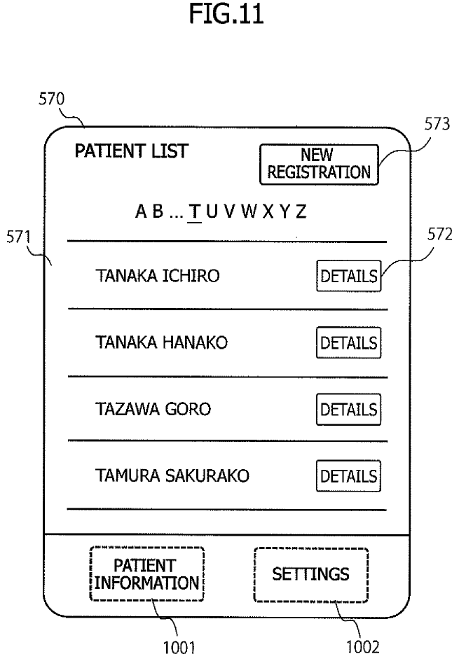

FIG. 11 illustrates an example of a patient list screen 570 that is one of the basic screens indicating the basic functions described above and is displayed by the patient information setting unit 103. Similarly to the settings screen 500, the patient list screen 570 displays a patient information screen link 1001 and a settings screen link 1002 at the bottom of the screen. The patient list screen 570 also displays a patient list 571 that is a list of patient names in patient information stored in the patient information storage unit 113 (S31 in FIG. 10). Each row of patient information in the patient list 571 displays a details button 572 for referring to the details of the corresponding patient information. Also, the patient list screen 570 displays a new registration button 573 for registering new patient information. In this specification, descriptions of the function of the patient information setting unit 103 for newly registering patient information are omitted.

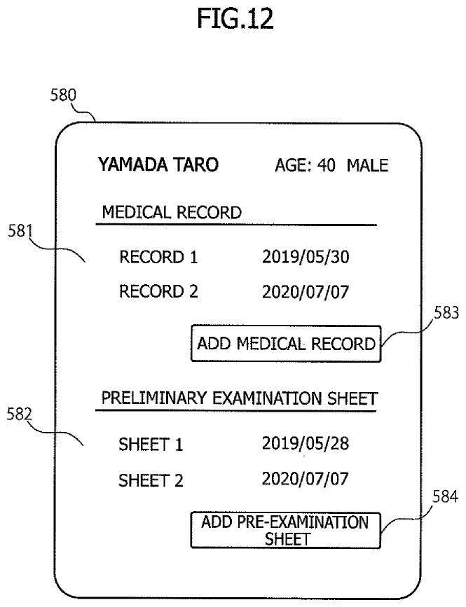

When the details button 572 in the patient list 571 is pressed by the user, the patient information setting unit 103 displays a patient details screen 580 illustrated as an example in FIG. 12 (S32). Also, the patient information setting unit 103 reads patient information from the patient information storage unit 113 and displays the read patient information on the patient details screen 580 (S33). Although only name, age, and sex are displayed as an example of patient information in FIG. 12, more detailed information may be displayed. Also, the patient information setting unit 103 reads prestored electronic medical record information from the electronic medical record storage unit 114 and displays the read electronic medical record information as an electronic medical record list 581 (S34). By specifying each row representing an electronic medical record in the electronic medical record list 581 by an operation such as tapping, it is possible to view detailed information of the electronic medical record and to update or delete the electronic medical record as necessary. Furthermore, the patient information setting unit 103 reads prestored information of preliminary examination sheets from the preliminary examination sheet storage unit 115 and displays the preliminary examination sheets as a preliminary examination sheet list 582 (S35). By specifying each preliminary examination sheet in the preliminary examination sheet list 582 by an operation such as tapping, it is possible to view detailed information of the preliminary examination sheet and to update or delete the preliminary examination sheet as necessary.

### 2.2.2. Electronic Medical Record Setting Process

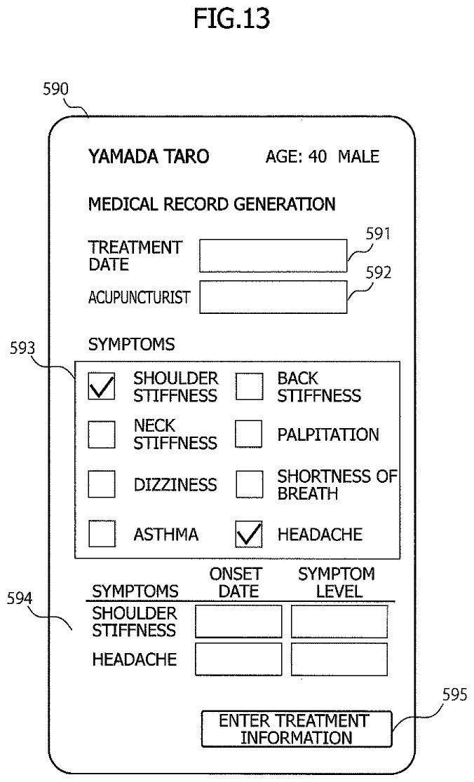

When a medical record addition button 583 is pressed by the user on the patient details screen 580, the electronic medical record setting unit 104 displays an electronic medical record setting screen 590 illustrated as an example in FIG. 13 (S41 in FIG. 10). The electronic medical record setting screen 590 displays, in addition to the patient information, input fields for a treatment date 591 and a staff member 592. The electronic medical record setting screen 590 also displays an input field for symptoms 593. The acupuncturist selects, from the symptoms 593, one or more symptoms that match the symptoms of the patient. As a result, the selected symptoms are displayed as a symptom list 594 below the symptoms 593. In the symptom list 594, the acupuncturist can enter an onset date and a symptom level for each symptom of the patient. Here, these information items related to symptoms can be entered by using information on symptoms set in a preliminary examination sheet that is stored in the preliminary examination sheet storage unit 115 by the preliminary examination sheet setting unit 106. The electronic medical record setting screen 590 further displays a treatment information entry button 595 for entering treatment information.

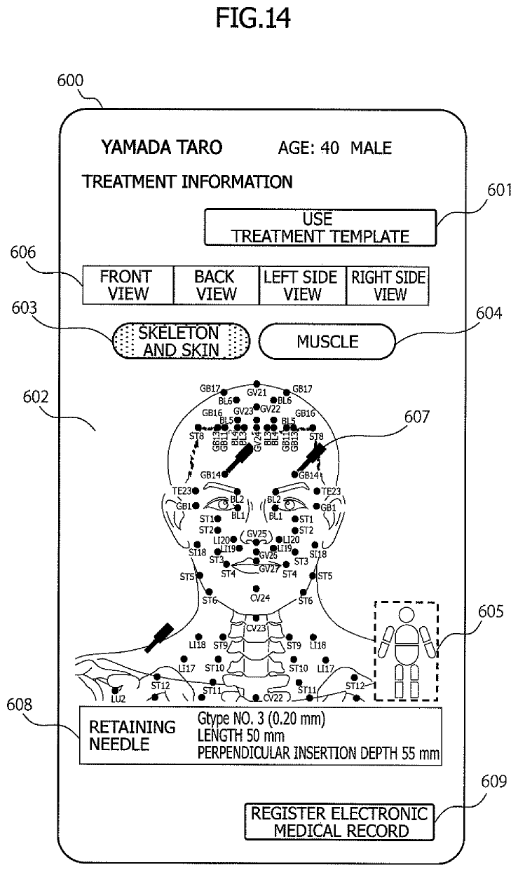

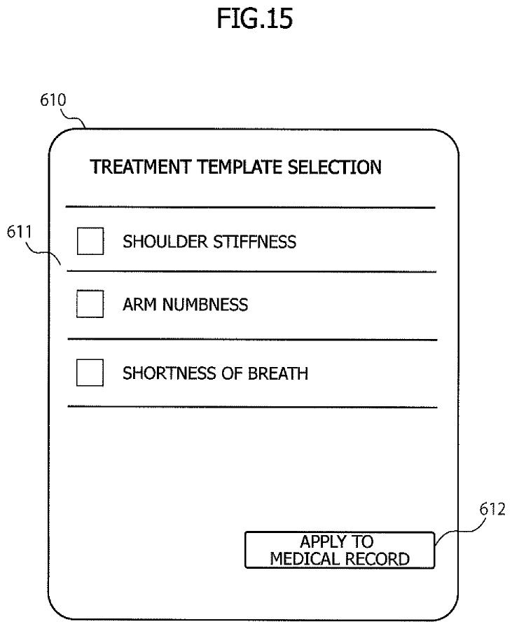

When the treatment information entry button 595 on the electronic medical record setting screen 590 is pressed by the user, the electronic medical record setting unit 104 displays a treatment information setting screen 600 illustrated as an example in FIG. 14 (S42). The treatment information setting screen 600 displays, in addition to the patient information, a “Use Treatment Template” button 601 for using, as treatment information, a treatment template that has already been generated. When the “Use Treatment Template” button 601 is pressed by the acupuncturist, the treatment template acquisition unit 105 displays a treatment template selection screen 610 illustrated as an example in FIG. 15 (S43). At this step, the treatment template acquisition unit 105 reads at least treatment template names of treatment templates from the treatment template storage unit 112 and displays a list of the read treatment template names as treatment template selection candidates 611 on the treatment template selection screen 610 (S44). The acupuncturist selects, in the treatment template selection candidate 611, the check box of a treatment template that matches a symptom of the patient for which an electronic medical record is to be generated, and presses an “Apply to Medical Record” button 612. In response, the treatment template acquisition unit 105 reads and obtains the selected treatment template from the treatment template storage unit 112. Then, the electronic medical record setting unit 104 displays the treatment information setting screen 600 again and displays, in a treatment input field 602, treatment information of the treatment template obtained by the treatment template acquisition unit 105 (S45).

Here, on the treatment template selection screen 610, it is also possible to select the check boxes of multiple treatment templates in the treatment template selection candidates 611. For example, in the example illustrated in FIG. 15, when symptoms of the patient include shoulder stiffness and arm numbness, the acupuncturist can select the check boxes of both of shoulder stiffness and arm numbness. In this case, the treatment template acquisition unit 105 obtains multiple treatment templates. Then, the electronic medical record setting unit 104 combines multiple sets of treatment information of the obtained treatment templates and superimposes the combined treatment information on the treatment input field 602 of the treatment information setting screen 600. When the same treatment site is included in multiple sets of treatment information of different treatment templates, the treatment information of one of the treatment templates is used and displayed for the treatment site.

Also, it is possible to display, in the treatment template selection candidates 611, only treatment templates corresponding to the symptoms of the patient, that is, only treatment templates having treatment template names including symptoms to which the treatment templates are applied and that match (or conform to) the symptoms entered on the electronic medical record setting screen 590. In this case, it is necessary to set symptoms in the treatment template names in such a format that the symptoms can be matched with symptoms entered when setting an electronic medical record. Also, when there is only one treatment template that matches the symptoms of the patient, the treatment template may be automatically selected and the treatment information of the treatment template may be directly applied to and displayed in the treatment input field 602 of the treatment information setting screen 600 without displaying the treatment template selection screen 610.

Similarly to the treatment template setting screen 540 described above, the treatment information setting screen 600 includes a skeleton and skin button 603, a muscle button 604, a human body mark 605, and display orientation switching buttons 606; and the display of the human figure can be changed by operating these buttons. Also, similarly to the treatment template setting screen 540, when the acupuncturist specifies a new treatment site on the human figure by an operation such as tapping, an icon 607 of “retaining needle” is displayed at the treatment site as a default value. Then, one of multiple sets of acupuncture and moxibustion information stored in the acupuncture and moxibustion information storage unit 111 is displayed as default values in an acupuncture and moxibustion information field 608 at the bottom of the screen. Also, when, for example, the icon 607 displayed at a treatment site to which a treatment template is applied is specified by an operation such as tapping, the acupuncture and moxibustion information set for the treatment site is displayed. When it is desired to set, in the treatment information, a procedure or acupuncture and moxibustion information different from the default values and the set values, the acupuncturist taps the acupuncture and moxibustion information field 608. In response, the electronic medical record setting unit 104 displays an acupuncture and moxibustion information selection screen 620 (S46). Because the acupuncture and moxibustion information selection screen 620 is the same as the acupuncture and moxibustion information selection screen 550, illustration of the acupuncture and moxibustion information selection screen 620 is omitted. The electronic medical record setting unit 104 reads acupuncture and moxibustion information stored in the acupuncture and moxibustion information storage unit 111 and displays the read acupuncture and moxibustion information on the acupuncture and moxibustion information selection screen 620 (S47); and then returns to the treatment information setting screen 600 to display selected acupuncture and moxibustion information (S48). Also, in response to a user operation on the acupuncture and moxibustion information selection screen 620, the electronic medical record setting unit 104 displays an acupuncture and moxibustion information individual setting screen 630 (S49). Because the acupuncture and moxibustion information individual setting screen 630 is also the same as the acupuncture and moxibustion information setting screen 520, illustration of the acupuncture and moxibustion information individual setting screen 630 is omitted. Then, the electronic medical record setting unit 104 returns to the treatment information setting screen 600 to display information entered on the acupuncture and moxibustion information individual setting screen 630 (S50).

When the acupuncturist completes all entries on the treatment information setting screen 600 and presses an electronic medical record registration button 609, the electronic medical record setting unit 104 generates an electronic medical record and stores the generated electronic medical record in the electronic medical record storage unit 114 (S51). The electronic medical record includes, in association with patient information, a treatment date, a staff member, symptoms (including onset dates and symptom levels), treatment sites, and procedures and acupuncture and moxibustion information for the respective treatment sites that are entered on the electronic medical record setting screen 590 and the treatment information setting screen 600 (and may also include information entered on the treatment template selection screen 610, the acupuncture and moxibustion information selection screen 620, and the acupuncture and moxibustion information individual setting screen 630).

### 2.2.3. Preliminary Examination Sheet Setting Process

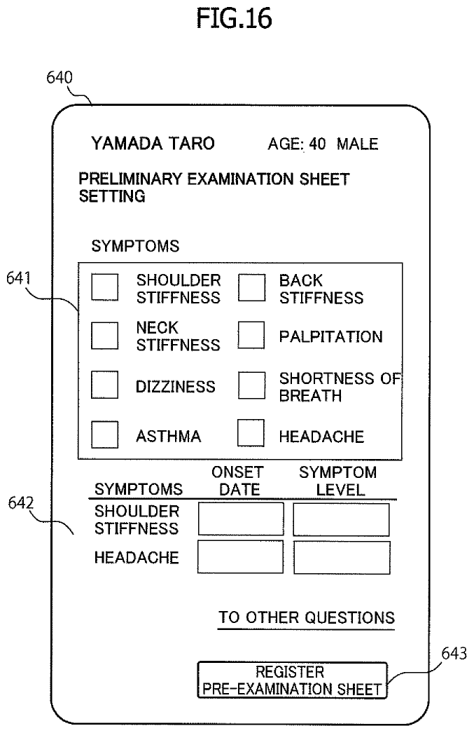

When a preliminary examination sheet addition button 584 is pressed by the acupuncturist on the patient details screen 580, the preliminary examination sheet setting unit 106 displays a preliminary examination sheet setting screen 640 illustrated as an example in FIG. 16 (S52). The preliminary examination sheet setting screen 640 displays, in addition to patient information, an input field for symptoms 641. The acupuncturist selects, from the symptoms 641, one or more symptoms that match the symptoms of the patient. As a result, similarly to the electronic medical record setting screen 590, the selected symptoms are displayed as a symptom list 642 below the symptoms 641. In the symptom list 642, the acupuncturist can enter an onset date and a symptom level for each symptom. Also, more detailed information (for example, medical history, lifestyle, etc.) can be entered on another screen by tapping a link “To Other Questions” on the preliminary examination sheet setting screen 640. However, descriptions of this function are omitted in this specification.

When a preliminary examination sheet registration button 643 on the preliminary examination sheet setting screen 640 is pressed by the acupuncturist, the preliminary examination sheet setting unit 106 stores, in the preliminary examination sheet storage unit 115, information such as symptoms (including onset dates and symptom levels) entered on the preliminary examination sheet setting screen 640, in association with patient information (S52).

### 3. Effects and Variations of Present Embodiment

As described above, the first embodiment makes it possible, when providing acupuncture and moxibustion treatment to a patient, to select a treatment template corresponding to a symptom of the patient from treatment templates that include symptoms to be treated by acupuncture and moxibustion and treatment information indicating treatment sites for the symptoms and procedures to be performed at the treatment sites, and to apply the selected treatment template to an electronic medical record. Accordingly, the first embodiment enables even an acupuncturist with relatively little experience to efficiently and accurately identify a treatment method in acupuncture and moxibustion treatment, and thereby makes it possible to improve accuracy of treatment. Furthermore, the first embodiment enables an acupuncturist to enter and set a treatment template. This not only enables an acupuncturist to use a treatment template set beforehand by the acupuncturist to provide treatment to a patient, but also enables an acupuncturist to use treatment templates set by other experienced acupuncturists to provide treatment to a patient.

Also, because it is possible to apply treatment information included in a treatment template to an electronic medical record as described above, the entry work of an electronic medical record during treatment can be greatly reduced. In the present embodiment, even when a treatment template is applied to an electronic medical record as described above, it is possible to add and modify treatment information on the treatment information setting screen 600 according to the specific treatment of each patient. This in turn makes it possible to set accurate treatment information tailored to each individual patient in an electronic medical record while reducing entry work by using a treatment template.

Also, as described above, when only treatment templates, in which symptoms matching the symptoms of a patient are set as the symptoms to which the treatment templates are applied, are displayed as the treatment template selection candidates 611, an acupuncturist can more easily select a treatment template suitable for the treatment of the patient. In this case, when there is only one treatment template that matches the symptoms of the patient, the treatment information of the treatment template may be directly applied to an electronic medical record to further facilitate the use of the treatment template.

Here, a procedure and acupuncture and moxibustion information corresponding to the procedure may be set in treatment information of a treatment template as necessary. For example, the acupuncture and moxibustion information may include, depending on the procedure, details of an acupuncture and moxibustion product such as the type, the number (thickness), and the length of the acupuncture and moxibustion product and also an angle and a depth in the procedure using the acupuncture and moxibustion product. This makes it possible to make treatment information indicated by a treatment template more specific and to improve the reproducibility of treatment when the treatment is performed according to the treatment template. Accordingly, an acupuncturist using a treatment template can perform more accurate treatment. Also, according to the present embodiment, acupuncture and moxibustion information as described above can be set when an electronic medical record is generated. This makes it possible to keep a detailed treatment record about what type of treatment was performed using what type of an acupuncture and moxibustion product, and to refer to the treatment record when performing the treatment the next time. Here, depending on the operation, acupuncture and moxibustion information may not be set, or only a some of the acupuncture and moxibustion information may be set. Furthermore, items in acupuncture and moxibustion information are not limited to those described above, and any item may be additionally set.

Here, when, for example, a site other than an acupuncture point is specified as a treatment site on the treatment template setting screen 540 used to set a treatment template, or on the treatment information setting screen 600 used to set an electronic medical record, an acupuncture point closest to the specified site other than an acupuncture point may be displayed as a candidate for a treatment site and the acupuncture point may be treated as a specified treatment site. In addition, all acupuncture points at distances less than a predetermined value from the specified site other than an acupuncture point may be displayed as options, and an acupuncture point selected from the options may be treated as a specified treatment site.

Also, when a site other than an acupuncture point is specified as a treatment site on the treatment template setting screen 540 or the treatment information setting screen 600 as described above, acupuncture points may be displayed in different manners depending on the distances from the specified site to the acupuncture points (for example, when a site is specified as a treatment site, acupuncture points existing within 1 cm from the site may be displayed in red and acupuncture points existing within 3 cm from the site may be displayed in blue, or acupuncture points may be listed for their respective distances, such as up to 3 cm and up to 5 cm, from the specified site).

Furthermore, for example, a function to display a list of acupuncture points specified as treatment sites on the treatment template setting screen 540 or the treatment information setting screen 600 may also be added. For example, acupuncture points that have already been specified during a process of entering treatment information in a treatment template or an electronic medical record may be displayed as the list of acupuncture points; or when referring to a treatment template or an electronic medical record that has already been generated and stored in a storage unit, all or a part of acupuncture points set in the treatment template or the electronic medical record may be displayed as the list of acupuncture points.

Also, the present embodiment enables an acupuncturist to store frequently used procedures and acupuncture and moxibustion information in the acupuncture and moxibustion information storage unit 111 and to select the stored procedures and acupuncture and moxibustion information when entering a treatment template or an electronic medical record. This makes it possible to reduce the load and simplify the work of entering a treatment template and an electronic medical record and to also reduce input errors in acupuncture and moxibustion information.

Treatment templates set as described above may be sold to, or shared with, for example, other acupuncture and moxibustion clinics (acupuncturists) or participants in acupuncture and moxibustion treatment seminars. This makes it possible to further improve the accuracy of acupuncture and moxibustion treatment in the entire industry.

Also, although an electronic medical record is entered by using a treatment template in the present embodiment, treatment information set in an electronic medical record may be applied to a treatment template. Specifically, for example, a button for registering treatment information in the treatment input field 602 and the acupuncture and moxibustion information field 608 as a treatment template may be provided on the treatment information setting screen 610 displayed by the electronic medical record setting unit 114. When the button is pressed by the acupuncturist, the treatment template setting unit 102 may display a user interface for entering a treatment template name and store treatment information entered on the treatment information setting screen 600 for an electronic medical record in the treatment template storage unit 112 in association with the treatment template name.

Furthermore, as a variation of the present embodiment, inventory information of acupuncture and moxibustion products in an acupuncture and moxibustion clinic may be stored in a storage unit, and the number of units of each acupuncture and moxibustion product indicated by acupuncture and moxibustion information (i.e., the number of units of each product used) in an electronic medical record generated at the acupuncture and moxibustion clinic may be automatically deducted from the inventory of the acupuncture and moxibustion product of the acupuncture and moxibustion clinic stored in advance in the storage unit. In this case, when multiple treatment templates are selected and used to set an electronic medical record and the same treatment site is included in multiple sets of treatment information of the multiple treatment templates, for this treatment site, only the number of units of an acupuncture and moxibustion product in the treatment information of one of the multiple treatment templates may be deducted from the inventory. Also, the production lot numbers of acupuncture needles may be stored and managed. This configuration eliminates the need to separately input a decrease in inventory to manage the inventory of acupuncture and moxibustion products at an acupuncture and moxibustion clinic and makes it possible to update inventory information by just entering electronic medical record information. Also, an average number of units of each acupuncture and moxibustion product used in a predetermined period may be calculated based on the number of units of the acupuncture and moxibustion product used, and a timing at which the stock of the acupuncture and moxibustion product becomes depleted may be predicted based on the average number of units of the acupuncture and moxibustion product used. Furthermore, the delivery time of each acupuncture and moxibustion product may be set in advance, and a warning may be output to urge the user to place an order at a timing obtained by deducting the delivery time from the timing at which the stock becomes depleted.

### Second Embodiment

### 1. Overall Configuration

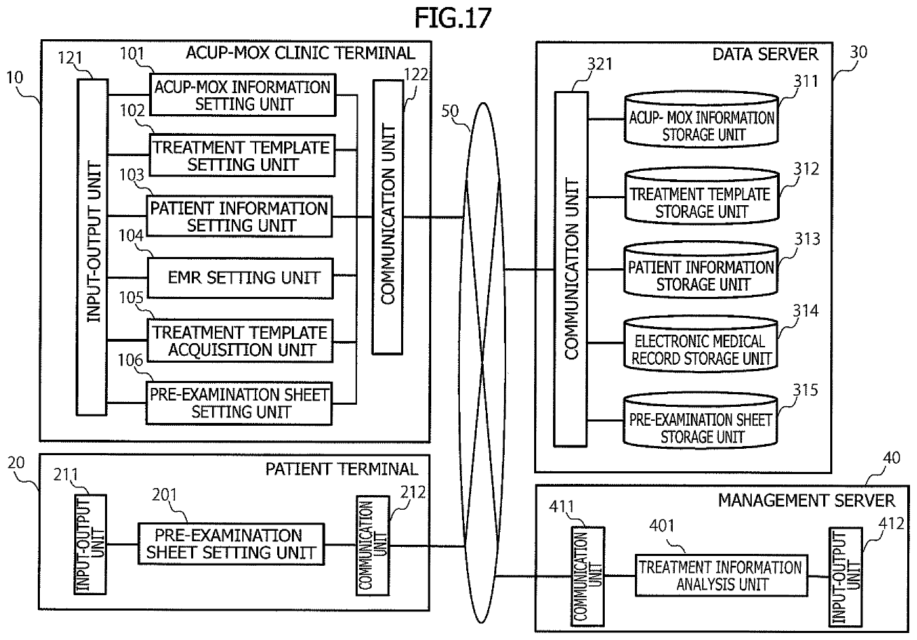

FIG. 17 illustrates an example of the overall configuration of an acupuncture and moxibustion treatment support system according to a second embodiment. The acupuncture and moxibustion treatment support system includes an acupuncture and moxibustion clinic terminal 10, a patient terminal 20, a data server 30, and a management server 40. Each of the acupuncture and moxibustion clinic terminal 10, the patient terminal 20, the data server 30, and the management server 40 is an information processing apparatus and may be implemented by, for example, a desktop computer, a mobile computer, a tablet computer, a mobile device, or any other type of device. The acupuncture and moxibustion clinic terminal 10, the patient terminal 20, the data server 30, and the management server 40 are interconnected via a network 50. The network 50 may include, for example, one or more of a local area network (LAN), a wide area network (WAN), the Internet, and the like.

In the second embodiment, storage units provided in the storage device of the acupuncture and moxibustion clinic terminal 10 in the first embodiment are provided in a storage device of the data server 30. Also, preliminary examination sheet information can be set not only by using the acupuncture and moxibustion clinic terminal 10, but also by using the patient terminal 20. Furthermore, the management server 40 can also access information in the storage units of the data server 30. Below, only configurations different from the first embodiment are described in detail.

The acupuncture and moxibustion clinic terminal 10 is used at an acupuncture and moxibustion clinic that provides acupuncture and moxibustion treatment. In the acupuncture and moxibustion treatment support system of the present embodiment, not only a case in which one acupuncture and moxibustion clinic terminal 10 is used, but also a case in which acupuncture and moxibustion clinic terminals 10 are used at multiple acupuncture and moxibustion clinics (and in addition, a case in which multiple acupuncture and moxibustion clinic terminals 10 are used in one acupuncture and moxibustion clinic) is assumed, and multiple acupuncture and moxibustion clinic terminals 10 may be present. The acupuncture and moxibustion clinic terminal 10 includes an acupuncture and moxibustion information setting unit 101, a treatment template setting unit 102, a patient information setting unit 103, an electronic medical record setting unit 104, a treatment template acquisition unit 105, and a preliminary examination sheet setting unit 106 the functions of which are implemented by executing an acupuncture and moxibustion treatment support program on the acupuncture and moxibustion clinic terminal 10. The acupuncture and moxibustion clinic terminal 10 also includes an input-output unit 121 and a communication unit 122.

The main functions of the acupuncture and moxibustion information setting unit 101, the treatment template setting unit 102, the patient information setting unit 103, the electronic medical record setting unit 104, the treatment template acquisition unit 105, and the preliminary examination sheet setting unit 106 are substantially the same as those of the first embodiment. The second embodiment is different from the first embodiment in that each of these units accesses, via the communication unit 122, an acupuncture and moxibustion information storage unit 311, a treatment template storage unit 312, a patient information storage unit 313, an electronic medical record storage unit 314, and a preliminary examination sheet storage unit 315 provided in the data server 30.

Also, in the second embodiment, multiple acupuncture and moxibustion clinic terminals 10 may be used in different acupuncture and moxibustion clinics. For this reason, when writing any type of information into the corresponding storage unit of the data server 30, each of the acupuncture and moxibustion information setting unit 101, the treatment template setting unit 102, the patient information setting unit 103, the electronic medical record setting unit 104, the treatment template acquisition unit 105, and the preliminary examination sheet setting unit 106 adds, to the information, acupuncture and moxibustion clinic information for identifying the acupuncture and moxibustion clinic by which the information is set. Here, basically, the information is accessible only from the acupuncture and moxibustion clinic by which the information is set. However, for example, it is also possible to enable the acupuncture and moxibustion clinic terminal 10 of another acupuncture and moxibustion clinic to access a certain piece of information as necessary by setting, in the information, an access control flag in which the other acupuncture and moxibustion clinic is specified.

The input-output unit 121 is the same as that of the first embodiment, and therefore, the descriptions of the input-output unit 121 are omitted here. The communication unit 122 is a communication module that functions as an interface for enabling communications between the acupuncture and moxibustion clinic terminal 10 and the network 50 and is implemented by, for example, a modem or a LAN card.

The patient terminal 20 is used by a patient who receives acupuncture and moxibustion treatment. In the acupuncture and moxibustion treatment support system of the present embodiment, it is also assumed that the patient terminal 20 is used by multiple patients, and there may be multiple patient terminals 20. For example, the patient terminal 20 may be a smartphone owned by the patient. The patient terminal 20 includes a preliminary examination sheet setting unit 201, the function of which is implemented by executing a part of the acupuncture and moxibustion treatment support program on the patient terminal 20. Also, the patient terminal 20 includes an input-output unit 211 and a communication unit 212.

The preliminary examination sheet setting unit 201 has functions similar to the functions of the preliminary examination sheet setting unit 106 of the acupuncture and moxibustion clinic terminal 10 described in the first embodiment, except that the user is a patient and the preliminary examination sheet is stored in a storage unit of the data server 30.

The input-output unit 210 is implemented by, for example, various drivers that function as interfaces with an input device and an output device provided in the patient terminal 20. The communication unit 211 is a communication module that functions as an interface for enabling communication between the patient terminal 20 and the network 50.

The data server 30 is an information processing apparatus that stores, in a storage device (storage unit), information written into the storage device by the acupuncture and moxibustion clinic terminal 10 and the patient terminal 20. The data server 30 includes the acupuncture and moxibustion information storage unit 311, the treatment template storage unit 312, the patient information storage unit 313, the electronic medical record storage unit 314, and the preliminary examination sheet storage unit 315 in a storage device provided in the data server 30. The data server 30 also includes a communication unit 321. The acupuncture and moxibustion clinic terminal 10, the patient terminal 20, and the management server 40 access the storage device as necessary via the communication unit 321 of the data server 30 and a module (not shown) running on the data server 30 and for reading and writing data from and into the storage device, and thereby read data from, and write data to, the acupuncture and moxibustion information storage unit 311, the treatment template storage unit 312, the patient information storage unit 313, the electronic medical record storage unit 314, and the preliminary examination sheet storage unit 315. The structures of data stored in the respective storage units are substantially the same as the structures of data stored in the acupuncture and moxibustion information storage unit 111, the treatment template storage unit 112, the patient information storage unit 113, the electronic medical record storage unit 114, and the preliminary examination sheet storage unit 115 in the first embodiment, except that each piece of data includes acupuncture and moxibustion clinic information and an access control flag in which one or more acupuncture and moxibustion clinics are specified.

The communication unit 321 is a communication module that functions as an interface for enabling communication between the data server 30 and the network 50.

The management server 40 can access the storage device of the data server 30. The management server 40 is mainly used by a system administrator of the acupuncture and moxibustion treatment support system. Here, a system administrator not only indicates a person who maintains and manages the operation of the system, but may also indicate any person (for example, a provider of acupuncture and moxibustion products) who is allowed to use information stored in the storage device of the data server 30. The management server 40 includes a treatment information analysis unit 401, the function of which is implemented by executing a part of the acupuncture and moxibustion treatment support program on the management server 40. The management server 40 also includes a communication unit 411 and an input-output unit 412.

The treatment information analysis unit 401 accesses the data server 30 via the communication unit 411 and analyzes contents of treatment provided by an acupuncture and moxibustion clinic based on treatment information included in at least one of a treatment template stored in the treatment template storage unit 312 and electronic medical record information stored in the electronic medical record storage unit 314, and outputs an analysis result via an output unit. The output unit is, for example, a display or a printer.

The communication unit 411 is a communication module that functions as an interface for enabling communications between the management server 40 and the network 50. The input-output unit 412 is implemented by, for example, various drivers that function as interfaces with an input device and an output device provided in the management server 40.

### 2. Treatment Information Analysis Process in Management Server

Next, the process performed in the acupuncture and moxibustion information support system of the second embodiment is described. Processes performed in the acupuncture and moxibustion clinic terminal 10 and the patient terminal 20 are substantially the same as the processes described in the first embodiment, and therefore, their descriptions are omitted here. In the second embodiment, an example of the process performed by the treatment information analysis unit 401 of the management server 40 is described with reference to examples of screens.

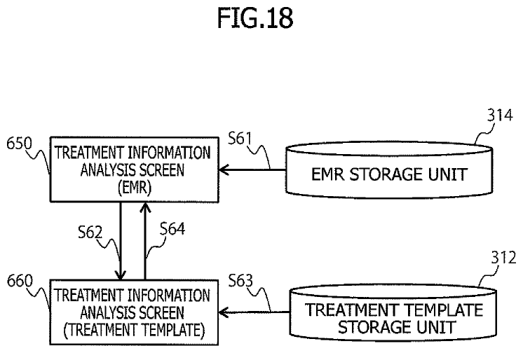

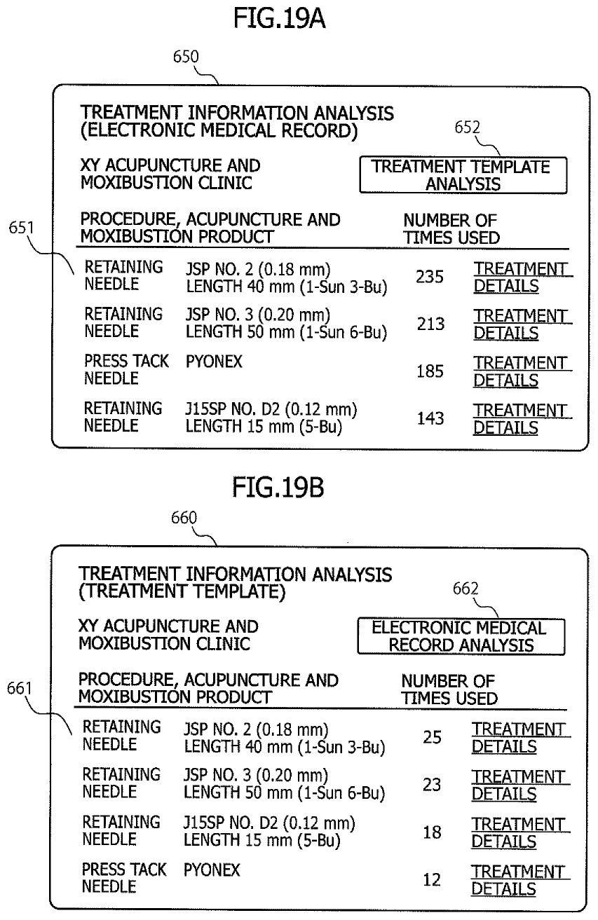

FIG. 18 is a drawing illustrating an example of a process performed by the treatment information analysis unit 401 along with displayed screens and access to storage units. FIG. 19 illustrates an example of a treatment information analysis screen (electronic medical record) 650 displayed by the treatment information analysis unit 401. The treatment information analysis screen (electronic medical record) 650 is displayed, for example, when a program is executed by an administrator. The treatment information analysis unit 401 accesses the electronic medical record storage unit 314 of the data server 30 to read information on electronic medical records stored in the electronic medical record storage unit 314, performs an analysis based on at least one of symptoms, treatment sites, procedures, and acupuncture and moxibustion information included in the electronic medical records, and outputs an analysis result (S61 in FIG. 18).

For example, as shown in an analysis result 651 of the treatment information analysis screen (electronic medical record) 650 illustrated as an example in FIG. 19A, the treatment information analysis unit 401 reads all electronic medical records of a given acupuncture and moxibustion clinic, and based on procedures and acupuncture and moxibustion information included in the electronic medical records, calculates and outputs information indicating how many times and for what procedure each acupuncture and moxibustion product was used, in other words, the number of units of each acupuncture and moxibustion product (each item) used. With this information, for example, a provider (seller) of acupuncture and moxibustion products can know what types of acupuncture and moxibustion products are often used in the acupuncture and moxibustion clinic. Here, when a treatment detail link is tapped in the row of each acupuncture and moxibustion product, for example, information indicating a symptom and a treatment site for which the procedure and the acupuncture and moxibustion product was used may be further displayed. Also, such an analysis may be performed for a group of multiple acupuncture and moxibustion clinics instead of for each acupuncture and moxibustion clinic, and may also be performed for each acupuncturist based on the information on a staff member in the electronic medical record.

Also, when a treatment template analysis button 652 is pressed by the user on the treatment information analysis screen (electronic medical record) 650, the treatment information analysis unit 401 displays a treatment information analysis screen (treatment template) 660 illustrated in FIG. 19B (S62). At this step, the treatment information analysis unit 401 accesses the treatment template storage unit 312 of the data server 30 to read information on treatment templates stored in the treatment template storage unit 312, performs an analysis based on at least one of treatment sites, procedures, and acupuncture and moxibustion information included in the treatment templates, and outputs an analysis result to an analysis result 661 on the treatment information analysis screen (treatment template) 660 (S63). Also in this case, similarly to the example of analysis based on electronic medical record information described above, it is possible to read all treatment templates of a given acupuncture and moxibustion clinic, and calculate and output information indicating how many times and for what procedure each acupuncture and moxibustion product was used, in other words, the number of units of each acupuncture and moxibustion product used. With an analysis based on treatment templates as described above, it is also possible to predict acupuncture and moxibustion products that are highly likely to be used in the future without relying on past usage records. Here, when an electronic medical record analysis button 662 is pressed by the user on the treatment information analysis screen (treatment template) 660, the treatment information analysis screen (electronic medical record) 650 is displayed again.

### 3. Effects and Variations of Present Embodiment

With the acupuncture and moxibustion treatment support system of the second embodiment, it is possible to analyze and understand treatment provided at an acupuncture and moxibustion clinic by analyzing treatment information at the management server 40 based on electronic medical record information stored in the electronic medical record storage unit 314 of the data server 30 and treatment template information stored in the treatment template storage unit 312 of the data server 30. For example, as in the examples described above, a provider of acupuncture and moxibustion products can know what types of acupuncture and moxibustion products are often used at each acupuncture and moxibustion clinic by analyzing the usage of each acupuncture and moxibustion product (for example, by calculating the number of units used) and thereby analyze types of acupuncture and moxibustion products that should be mainly sold to the acupuncture and moxibustion clinic.

The analysis described above is just an example, and the treatment information analysis unit 401 can perform any type of analysis as necessary by using data included in electronic medical record information and treatment template information. For example, even when acupuncture and moxibustion information is not stored, it is possible to analyze the correlation between symptoms of patients and treatment sites and procedures for the symptoms (i.e., what type of procedure is performed at which treatment site for a given symptom). Also, it is possible to analyze the correlation between treatment sites and procedures and acupuncture and moxibustion products (i.e., what type of procedure and what type of acupuncture and moxibustion product are used at a given treatment site (e.g., at a given acupuncture point)). Here, for example, when a site other than an acupuncture point is specified as a treatment site, an acupuncture point at the shortest distance from the treatment site may be identified, and an analysis may be performed assuming that the acupuncture point is specified as the treatment site. Also, because electronic medical record information includes treatment dates, analyses described above as examples may be performed as time-series analyses. For example, the number of units of an acupuncture and moxibustion product used may be calculated as a time series (for example, the number of units used may be calculated for each week or month) to identify changes in the number of units used. Also, such analyses may be performed for electronic medical record information and treatment templates of all acupuncture and moxibustion clinics in the data server 30 or may be performed, for example, for each acupuncture and moxibustion clinic or each acupuncturist in charge.

When performing an analysis for each acupuncture and moxibustion clinic or each acupuncturist, a treatment information analysis unit may be provided in the acupuncture and moxibustion clinic terminal 10 instead of in the management server 40, and only treatment information stored in a storage unit of the acupuncture and moxibustion clinic terminal 10 may be analyzed. In this case, for example, the analysis result may be output to an output unit provided in the acupuncture and moxibustion clinic terminal 10 via the input-output unit 121, or may be sent via the communication unit 122 to another device such as the management server 40.

It is also possible to provide a treatment information analysis unit in the data server 30 instead of in the management server 40 and to analyze treatment information based on information stored in the storage device of the data server 30. In this case, the data server 30 may output the analysis result to an output unit of the data server 30 or send the analysis result to another device.

Also, in the configuration described above as a variation of the first embodiment in which the number of units of an acupuncture and moxibustion product used in acupuncture and moxibustion information included in electronic medical record information registered at an acupuncture and moxibustion clinic is automatically deducted from the stock quantity in the inventory information of the acupuncture and moxibustion product in the acupuncture and moxibustion clinic, the inventory information of each acupuncture and moxibustion clinic may be stored in the data server 30, and the management server 40 may be configured to enable a provider of acupuncture and moxibustion products to refer to inventory information of acupuncture and moxibustion products in each acupuncture and moxibustion clinic and thereby determine how many acupuncture and moxibustion products need to be provided to the acupuncture and moxibustion clinic. Also, similarly to the first embodiment, the management server 40 may be configured to calculate an average number of units of each acupuncture and moxibustion product used in a predetermined period based on the number of units of the acupuncture and moxibustion product used and to predict, based on the average number of units of the acupuncture and moxibustion product used, a timing at which the stock of the acupuncture and moxibustion product in the acupuncture and moxibustion clinic will be depleted. Furthermore, the delivery time of each acupuncture and moxibustion product may be set in advance, and the management server 40 may be configured to perform a process of ordering the acupuncture and moxibustion product at a timing obtained by subtracting the delivery time from the timing at which the stock will be depleted.

Furthermore, because various types of information set by the acupuncture and moxibustion clinic terminal 10 are stored in the storage units of the data server 30 that are accessible from the acupuncture and moxibustion clinic terminals 10 of multiple acupuncture and moxibustion clinics, the information can be shared with other acupuncture and moxibustion clinics by using, for example, the access control flag described above. For example, a treatment template generated at an acupuncture and moxibustion clinic can be shared with other acupuncture and moxibustion clinics belonging to the same management group. Also, even when a patient changes clinics, it is possible to share electronic medical records with a destination acupuncture and moxibustion clinic.

Also, by requesting a patient to store a preliminary examination sheet in the preliminary examination sheet storage unit 315 from the patient terminal 20 and referring to the preliminary examination sheet using the acupuncture and moxibustion clinic terminal 10, the acupuncturist can understand the symptoms of the patient before the patient receives treatment at the acupuncture and moxibustion clinic and can consider treatment policy in advance. In this case, for example, it is also possible to consider a treatment template to be used. This enables the patient terminal 20 to set a preliminary examination sheet and makes it possible to reduce the work that needs to be performed when a patient visits a clinic.

In both of the first embodiment and the second embodiment, the acupuncture and moxibustion information setting unit 101, the treatment template setting unit 102, the patient information setting unit 103, the electronic medical record setting unit 104, the treatment template acquisition unit 105, and the preliminary examination sheet setting unit 106 are provided in the same acupuncture and moxibustion clinic terminal 10. However, all or some of these units may be distributed to different acupuncture and moxibustion clinic terminals 10. For example, a treatment template generated by the treatment template setting unit 102 of a first acupuncture and moxibustion clinic terminal 10 may be stored in the treatment template storage unit 112, and the treatment template may be used by accessing the treatment template storage unit 112 from the treatment template acquisition unit 105 provided in a second acupuncture and moxibustion clinic terminal 10. Similarly, each set of the acupuncture and moxibustion information storage unit 111, the treatment template storage unit 112, the patient information storage unit 113, the electronic medical record storage unit 114, and the preliminary examination sheet storage unit 115 in the first embodiment and the acupuncture and moxibustion information storage unit 311, the treatment template storage unit 312, the patient information storage unit 313, the electronic medical record storage unit 314, and the preliminary examination sheet storage unit 315 in the second embodiment may not necessarily be provided in the same device, and all or some of these units may be distributed to different devices.

### [Hardware Configuration of Information Processing Apparatus]

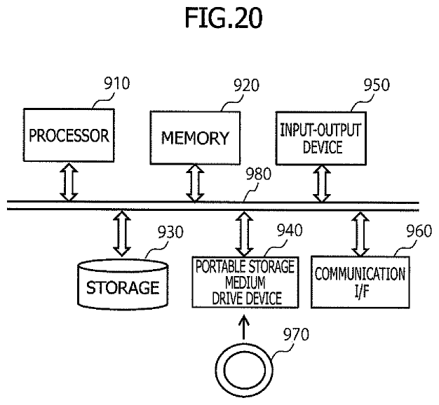

FIG. 20 illustrates an example of a hardware configuration of an information processing apparatus for each of the acupuncture and moxibustion clinic terminal 10, the patient terminal 20, the data server 30, and the management server 40, which are components of the acupuncture and moxibustion treatment support system in the first embodiment and the second embodiment described above. The information processing apparatus includes a processor 910, a memory 920, a storage 930, a portable storage medium drive device 940, an input-output device 950, and a communication interface 960.

The processor 910 includes a control unit, an arithmetic unit, and an instruction decoder. In the processor 910, an execution unit causes the arithmetic unit to perform arithmetic and logical operations according to instructions of a program decoded by the instruction decoder in response to a control signal output from the control unit. The processor 910 also includes a control register that stores various types of information used for control and a cache that can temporarily store information obtained from, for example, the memory 2 that has already been accessed. The processor 910 may have a configuration that includes multiple central processing unit (CPU) cores.

The memory 920 is, for example, a storage device such as a random-access memory (RAM) and is a main memory into which programs executed by the processor 910 are loaded and that stores data used in processes performed by the processor 910. The storage 930 is a storage device such as a hard disk drive (HDD) or a flash memory, and stores programs and various types of data. The portable storage medium drive device 940 reads data and programs stored in a portable storage medium 970. The portable storage medium 970 is, for example, a magnetic disk, an optical disk, a magneto-optical disk, or a flash memory. The processor 910 executes programs stored in the storage 930 and the portable storage medium 970 in corporation with the memory 920 and the storage 930. Here, programs to be executed and data to be accessed by the processor 910 may be stored in another device that can communicate with the information processing apparatus.

The input-output device 950 is, for example, a touch panel, a keyboard, and/or a display, receives operation commands corresponding to operations performed by the user, and outputs results of processes performed by the information processing apparatus. The communication interface 960 is, for example, a communication module such as the communication unit 122 of the acupuncture and moxibustion clinic terminal 10 in the second embodiment described above, and enables data communications with external devices. The components of the information processing apparatus described above are connected to each other via a bus 980.

### [Other Matters]

The functional configuration and the physical configuration of the information processing apparatus described in this specification are not limited to those described above. For example, functions and physical resources described above may be integrated or conversely, may be further distributed, for implementation.

### REFERENCE SYMBOL LIST

10 . . . acupuncture and moxibustion clinic terminal; 101 . . . acupuncture and moxibustion information setting unit; 102 . . . treatment template setting unit; 103 . . . patient information setting unit; 104 . . . electronic medical record setting unit; 105 . . . treatment template acquisition unit; 106, 201 . . . preliminary examination sheet setting unit; 111, 311 . . . acupuncture and moxibustion information storage unit; 112, 312 . . . treatment template storage unit; 113, 313 . . . patient information storage unit; 114, 314 . . . electronic medical record storage unit; 115, 315 . . . preliminary examination sheet storage unit; 20 . . . patient terminal; 30 . . . data server; 40 . . . management server

1. An acupuncture and moxibustion treatment support apparatus comprising:
a treatment template acquisition unit that reads and obtains, from a storage unit, a treatment template that is selected based on a symptom of a patient among treatment templates in each of which a symptom to be treated by acupuncture and moxibustion treatment is associated with treatment information indicating a treatment site for the symptom and a procedure to be performed at the treatment site; and
an electronic medical record setting unit that generates an electronic medical record of the patient to which the treatment information included in the obtained treatment template is applied and that stores the generated electronic medical record in the storage unit.

2. The acupuncture and moxibustion treatment support apparatus as claimed in claim 1, wherein the treatment template acquisition unit displays the treatment templates associated with the symptom of the patient on a user interface as selection candidates, and obtains the treatment information included in the treatment template selected from the selection candidates.

3. The acupuncture and moxibustion treatment support apparatus as claimed in claim 1, further comprising:
a treatment template setting unit that generates the treatment template based on input of the symptom to be treated by the acupuncture and moxibustion treatment and the treatment information, and stores the generated treatment template in the storage unit.

4. The acupuncture and moxibustion treatment support apparatus as claimed in claim 3, wherein the treatment template setting unit reads, from the storage unit, the electronic medical record stored in advance in the storage unit and generates, based on the treatment information included in the electronic medical record, the treatment template in which the symptom of the patient who is a subject of the electronic medical record is associated with the treatment information.

5. The acupuncture and moxibustion treatment support apparatus as claimed in claim 1, wherein the treatment information further includes acupuncture and moxibustion information that includes details of an acupuncture and moxibustion product used in the procedure.

6. The acupuncture and moxibustion treatment support apparatus as claimed in claim 5, wherein the acupuncture and moxibustion information includes at least any one of types of the acupuncture and moxibustion products, thicknesses of acupuncture needles, and lengths of the acupuncture needles.

7. The acupuncture and moxibustion treatment support apparatus as claimed in claim 6, wherein the acupuncture and moxibustion information further includes any of insertion angles or insertion depths of the acupuncture and moxibustion products in the procedure.

8. The acupuncture and moxibustion treatment support apparatus as claimed in claim 5, further comprising:
an acupuncture and moxibustion information setting unit that stores procedures and sets of acupuncture and moxibustion information corresponding to the procedures in the storage unit in advance,
wherein the treatment information includes acupuncture and moxibustion information selected from the sets of acupuncture and moxibustion information stored in the storage unit.

9. The acupuncture and moxibustion treatment support apparatus as claimed in claim 5, wherein when generating the electronic medical record, the electronic medical record setting unit deducts a number of units of the acupuncture and moxibustion product used from inventory of the acupuncture and moxibustion product stored in advance in the storage unit, the number of units of the acupuncture and moxibustion product used being indicated by the acupuncture and moxibustion information included in the treatment information in the electronic medical record.

10. The acupuncture and moxibustion treatment support apparatus as claimed in claim 9, wherein
when multiple treatment templates are selected and obtained, the electronic medical record setting unit integrates sets of treatment information included in the multiple treatment templates and applies the integrated treatment information to the electronic medical record; and
when the same treatment site is included in the sets of treatment information of the multiple treatment templates, for the treatment site, the electronic medical record setting unit deducts, from the inventory, only the number of units of the acupuncture and moxibustion product used in one of the sets of treatment information.

11. The acupuncture and moxibustion treatment support apparatus as claimed in claim 1, wherein the electronic medical record setting unit generates the electronic medical record including changed treatment information based on input to change the treatment information included in the treatment template read from the storage unit.

12. The acupuncture and moxibustion treatment support apparatus as claimed in claim 1, further comprising:
a treatment information analysis unit that analyzes contents of acupuncture and moxibustion treatment based on the treatment information included in at least one of the electronic medical record or the treatment template stored in the storage unit and outputs an analysis result.

13. The acupuncture and moxibustion treatment support apparatus as claimed in claim 12, wherein when a site other than an acupuncture point is specified as the treatment site in the treatment information, the treatment information analysis unit identifies an acupuncture point at a shortest distance from the treatment site, and analyzes the contents of acupuncture and moxibustion treatment assuming that the acupuncture point is specified as the treatment site.

14. An acupuncture and moxibustion treatment support method performed by an information processing apparatus, the method comprising:
reading and obtaining, from a storage unit, a treatment template that is selected based on a symptom of a patient among treatment templates in each of which a symptom to be treated by acupuncture and moxibustion treatment is associated with treatment information indicating a treatment site for the symptom and a procedure to be performed at the treatment site;
generating an electronic medical record of the patient to which the treatment information included in the obtained treatment template is applied; and
storing the generated electronic medical record in the storage unit.

15. A computer-readable, non-transitory medium storing an acupuncture and moxibustion treatment support program for causing a computer to execute a process including:
reading and obtaining, from a storage unit, a treatment template that is selected based on a symptom of a patient among treatment templates in each of which a symptom to be treated by acupuncture and moxibustion treatment is associated with treatment information indicating a treatment site for the symptom and a procedure to be performed at the treatment site;
generating an electronic medical record of the patient to which the treatment information included in the obtained treatment template is applied; and
storing the generated electronic medical record in the storage unit.

16. An acupuncture and moxibustion treatment support system comprising:
one or more acupuncture and moxibustion clinic terminals used in one or more acupuncture and moxibustion clinics that provide acupuncture and moxibustion treatment;
a data server including a storage unit that stores information written into the storage unit by the acupuncture and moxibustion clinic terminals; and
a management server that is capable of accessing the storage unit of the data server, wherein
each of the acupuncture and moxibustion clinic terminals reads and obtains, from the storage unit, a treatment template that is selected based on a symptom of a patient among treatment templates in each of which a symptom to be treated by acupuncture and moxibustion treatment is associated with treatment information indicating a treatment site for the symptom and a procedure to be performed at the treatment site, generates an electronic medical record of the patient to which the treatment information included in the obtained treatment template is applied, and stores the generated electronic medical record in the storage unit; and
the management server analyzes contents of acupuncture and moxibustion treatment based on the treatment information included in at least one of the electronic medical record and the treatment template stored in the storage unit and outputs an analysis result.
